(19) United States
(12) Patent Application Publication
(10) Pub. No.: US 2021/0318337 A1
(43) Pub. Date: Oct. 14, 2021

(54) Biomarkers for Precisely Predicting the Glomerular Filtration Rate and for Indicating Pathophysiologic Factors of an Impaired Glomerular Filtration Rate

(71) Applicant: numares AG, Regensburg (DE)

(72) Inventors: Eric SCHIFFER, Nürnberg (DE); Jana FRUTH, Dortmund (DE); Volker PFAHLERT, Kandern (DE); Maximilian ZUCKER, München (DE); Tobias STEINLE, Regensburg (DE); Johannes EIGLSPERGER, Walkenstetten (DE)

(21) Appl. No.: 17/271,675

(22) PCT Filed: Sep. 30, 2019
(86) PCT No.: PCT/EP2019/076417

§ 371 Date: Feb. 26, 2021

(30) Foreign Application Priority Data
Sep. 29, 2018 (DE) 10 2018 216 820.2

(51) Int. Cl. G01N 33/70; G01N 33/68

(52) U.S. Cl. CPC G01N 33/70; G01N 33/6893

(57) ABSTRACT

An in vitro method for calculating a glomerular filtration rate of an individual or an indicator of a pathophysiologic factor of an impaired glomerular filtration rate is provided. The method may include the following steps: providing a blood sample from an individual; determining the concentration of at least three substances chosen from alanine, choline, creatine, creatinine, dimethyl sulfone, dimethylamine, glucose, glycerol, isoleucine, leucine, myo-inositol, N,N-dimethylglycine, and valine in the blood sample by analyzing the blood sample with a suited measuring technique; and calculating a predicted glomerular filtration rate or an indicator of a pathophysiologic factor of an impaired glomerular filtration rate from the determined concentrations of the substances.

### CROSS-REFERENCE TO RELATED APPLICATIONS

This application is the United States national phase of International Application No. PCT/EP2019/076417 filed Sep. 30, 2019, and claims priority to German Patent Application No. 10 2018 216 820.2 filed Sep. 29, 2018, the disclosures of which are hereby incorporated by reference in their entirety.

### BACKGROUND OF THE INVENTION

### Field of the Invention

The disclosure relates to an in vitro method for calculating a glomerular filtration rate of an individual or for indicating a pathophysiologic reason of an impaired glomerular filtration rate.

### Description of Related Art

The glomerular filtration rate (GFR) is a measure of the volume of blood plasma filtered by the kidneys in a specific time. The GFR directly indicates the total volume of the primary urine that is formed by all glomeruli of both kidneys per time.

Measuring the GFR using renal or plasma clearance of an exogenous filtration marker (tracer) to obtain a measured GFR (mGFR) is the gold standard for assessing kidney function, but this procedure is time consuming and associated with a high burden for patients. Therefore, GFR is commonly estimated from serum creatinine (eGFR_{creat}) or serum cystatin C (eGFR_{cycC}). However, there exist different estimating equations for adult and pediatric patients or patients under specific treatments such as chemotherapy. These equations often perform only moderately outside the cohort in which they were developed.

WO 2016/025429 A1 describes a method for calculating the estimated glomerular filtration rate (eGFR) in a patient comprises the steps of (a) measuring the level of one or more metabolites using mass spectrometry from a blood sample obtained from the patient; and (b) calculating the eGFR using an algorithm that utilizes the measured levels of the one or more metabolites.

EP 3 309 554 A1 does not rely on calculating eGFR but describes calculating a disease-state index value for renal disorders on the basis of the quantities of D-form and/or L-form amino acids, from feces or intestinal content. By comparing the disease-state index value with a threshold value determined from the disease-state index values of a renal failure patient group and a healthy subject group, it is possible to diagnose a mild renal disorder patient group.

Generally, the higher the real GFR is, the bigger is the deviation between the estimated GFR (eGFR) and the real GFR.

If, however, the eGFR does not properly reflect the real GFR, the health status of an individual might be wrongly assessed. As a consequence, an incorrect diagnosis may be made or inappropriate therapies might be prescribed.

### SUMMARY OF THE INVENTION

Thus, there is a need in providing a possibility to calculate the GFR more exactly than according to the methods known from prior art.

This need is addressed by the novel method for using combinations of specific substances as biomarkers for calculating the GFR as described herein. The biomarkers to be used are at least three substances of the group consisting of alanine, choline, creatine, creatinine, dimethyl sulfone, dimethylamine, glucose, glycerol, isoleucine, leucine, myo-inositol, N,N-dimethylglycine, and valine. The method for calculating the GFR is an in vitro method. In an aspect of the proposed solution, these biomarkers can also be used in an in vivo method, i.e. in a medical method for determining the GFR, e.g., by analyzing blood of a patient during ongoing dialysis.

In an embodiment, exactly three biomarkers of this group are used. In an embodiment, at least or exactly four, in particular at least or exactly five, in particular at least or exactly six, in particular at least or exactly seven, in particular at least or exactly eight, in particular at least or exactly nine, in particular at least or exactly ten, in particular at least or exactly eleven, in particular at least or exactly twelve biomarkers of this group are used. In an embodiment, all listed biomarkers are combined with each other and used collectively.

Subgroups of at least three biomarkers in each case of the group of possible biomarkers listed above or all of these biomarkers cannot only be used for calculating the GFR. Alternatively or additionally, they are also very well suited to be used in an in vitro method for providing an indicator of a pathophysiologic factor (reason) of an impaired glomerular filtration rate. Thus, by using at least one subgroup of three biomarkers of the group listed above or all of these biomarkers, it is not only possible to determine the GFR much more exactly than known from prior art, but one additionally gains insight into the pathophysiologic reasons for an impaired GFR. Thus, the information provided in a single step by using these biomarkers is significantly more than just a further method for calculating the GFR.

The proposed solution has different properties. On the one hand, the calculated GFR matches the real GFR much better than GFRs calculated by models used in prior art. This will be explained in more detail with respect to the exemplary embodiments. On the other hand, besides simply calculating the GFR, information is provided why the GFR is impaired (lower than normal or higher than normal). This is particular interesting for decisions regarding the therapy of a patient having an impaired GFR. While patients with impaired GFR might have a clinically very similar appearance, the pathophysiologic reasons for the clinically diagnosed appearance of the patient might be completely different. Then, these patients should undergo different therapies in order to ameliorate their health status. According to the different prior art techniques of calculating the eGFR, it was not possible to draw further conclusions on any reasons for an impaired GFR. One simply obtained the information that the calculated GFR differs from the normal GFR.

The subgroup of at least three biomarkers of the group listed above can be likewise applied for calculating the GFR of an adult or of the GFR of a child. Thus, it is not necessary to choose different biomarkers for calculating the GFR of children. In contrast, the calculation methods used in prior art regularly make a distinction between calculation of the GFR for an adult or for a child. Furthermore, the novel combination of biomarkers is particularly appropriate for estimating the GFR of pregnant women where tracer based methods are not indicated.

In an embodiment, the group from which the biomarkers can be chosen consists of creatine, creatinine, dimethyl sulfone, glycerol, myo-inositol, and valine.

In an embodiment, the marker set comprises creatinine, dimethyl sulfone, and valine. A combination of these three biomarkers is particularly appropriate to calculate the GFR over a broad range (0 mL/min/1.73 m² to 150 mL/min/1.73 m²) in a simple manner.

In an embodiment, the marker set comprises creatinine, myo-inositol, and valine. A combination of these three biomarkers is also particularly appropriate to calculate the GFR over a broad range (0 mL/min/1.73 m² to 150 mL/min/1.73 m²) in a simple manner.

In an embodiment, the marker set does not comprise creatinine, leucine, myo-inositol, and valine at the same time. To give an example, the marker set might comprise in this embodiment two substances of creatinine, leucine, myo-inositol, and valine together with a third substance of the list of possible substances indicated above. To give another example, the marker set might comprise in this embodiment one substance of creatinine, leucine, myo-inositol, and valine together with a second and a third substance of the list of possible substances indicated above.

In an embodiment, the marker set does not comprise alanine, creatinine, leucine, myo-inositol, and valine at the same time. To give an example, the marker set might comprise in this embodiment two substances of alanine, creatinine, leucine, myo-inositol, and valine together with a third substance of the list of possible substances indicated above. To give another example, the marker set might comprise in this embodiment one substance of alanine, creatinine, leucine, myo-inositol, and valine together with a second and a third substance of the list of possible substances indicated above.

In an embodiment, the marker set does not exclusively comprise amino acids, but requires at least one substance being not an amino acid.

In an embodiment, the marker set comprises creatinine, dimethyl sulfone, myo-inositol, and valine. Some of these biomarkers are particularly appropriate to calculate the GFR over a range from 0 mL/min/1.73 m² to 90 mL/min/1.73 m², in particular from 10 mL/min/1.73 m² to 80 mL/min/1.73 m², in particular from 20 mL/min/1.73 m² to 70 mL/min/1.73 m², in particular from 30 mL/min/1.73 m² to 60 mL/min/1.73 m², whereas some of these biomarkers are particularly appropriate to calculate the GFR over a range from 60 mL/min/1.73 m² to 150 mL/min/1.73 m², in particular from 70 mL/min/1.73 m² to 140 mL/min/1.73 m², in particular from 80 mL/min/1.73 m² to 130 mL/min/1.73 m², in particular from 90 mL/min/1.73 m² to 120 mL/min/1.73 m². By combining these markers in a marker set and by combining equations making use of (overlapping) subsets of these markers, a particular precise calculation of the GFR over a range of 0 mL/min/1.73 m² to 150 mL/min/1.73 m² is possible.

In an embodiment, the marker set comprises creatine, creatinine, dimethyl sulfone, glycerol, and valine. Some of these biomarkers are particularly appropriate to calculate the GFR over a range from 0 mL/min/1.73 m² to 90 mL/min/1.73 m², in particular from 10 mL/min/1.73 m² to 80 mL/min/1.73 m², in particular from 20 mL/min/1.73 m² to 70 mL/min/1.73 m², in particular from 30 mL/min/1.73 m² to 60 mL/min/1.73 m², whereas some of these biomarkers are particularly appropriate to calculate the GFR over a range from 60 mL/min/1.73 m² to 150 mL/min/1.73 m², in particular from 70 mL/min/1.73 m² to 140 mL/min/1.73 m², in particular from 80 mL/min/1.73 m² to 130 mL/min/1.73 m², in particular from 90 mL/min/1.73 m² to 120 mL/min/1.73 m². By combining these markers in a marker set and by combining equations making use of (overlapping) subsets of these markers, a particular precise calculation of the GFR over a range of 0 mL/min/1.73 m² to 150 mL/min/1.73 m² is possible.

In an embodiment, the pathophysiologic factor determined by the novel biomarkers is chosen from the categories consisting of renal failure, renal co-morbidity, and extra-renal co-morbidity. Thereby, it is possible to determine pathophysiologic factors belonging to different categories.

Specific examples of the pathophysiologic factor determined in an embodiment are renal filtration, reabsorption and/or secretion capability, uremia, renal metabolic acidosis, renal oxidative stress, altered renal gluconeogenesis, amyotrophia, sarcopenia, changes of gut microbiome and/or tubular hyperosmolality. Thereby, it is possible to determine a single or more than one of these pathophysiologic factors, wherein any combination of the pathophysiologic factors is possible.

Altered renal filtration, reabsorption and/or secretion capability is an example of a pathophysiologic factor belonging to the group of renal failure. Altered concentrations of creatinine, alanine, glycine, valine, leucine, isoleucine, dimethyl sulfone, myo-inositol, N,N-dimethylglycine, and dimethylamine, are appropriate indicators for indicating an altered renal filtration, reabsorption and/or secretion capability. Thereby, an increased concentration of any of creatinine, alanine, glycine, dimethyl sulfone, myo-inositol, N,N-dimethylglycine, and dimethylamine is indicative for impaired renal filtration, renal reabsorption and/or renal secretion.

Likewise, a decreased concentration of leucine, isoleucine and/or of alanine is indicative for impaired renal filtration, renal reabsorption and/or renal secretion.

An example of renal co-morbidity is metabolic acidosis. The concentrations of valine, leucine and/or isoleucine are appropriate indicators for indicating the presence of such metabolic acidosis. Thereby, an increased concentration of any of these amino acids goes along with the presence of metabolic acidosis.

Another example of a factor causative for renal co-morbidity is oxidative stress. The concentrations of myo-inositol and dimethyl sulfone (used alone or in combination) are appropriate examples to indicate the presence of oxidative stress. Thereby, a decreased concentration of any of these substances goes along with the presence of oxidative stress.

Altered renal gluconeogenesis is another example of a factor being causative for renal co-morbidity. The concentration of alanine is an appropriate example of an indicator of altered renal gluconeogenesis. Thereby, a decreased concentration of alanine typically goes along with the presence of altered renal gluconeogenesis.

An example of extra-renal co-morbidity are changes of gut microbiome. The concentration of dimethyl sulfone and/or dimethylamine are appropriate examples of an indicator of such changes of gut microbiome. Thereby, a decreased concentration of these substances typically goes along with changes of gut microbiome.

Tubular hyperosmolality is another example of a factor being causative for extra-renal co-morbidity. The concentration of myo-inositol is an appropriate example of an indicator of tubular hyperosmolality. Thereby, a decreased concentration of myo-inositol typically goes along with the presence of tubular hyperosmolality.

An aspect of the proposed solution relates to the medical method for using of a marker set comprising at least three substances chosen from the group consisting of alanine, choline, creatine, creatinine, dimethyl sulfone, dimethylamine, glucose, glycerol, isoleucine, leucine, myo-inositol, N,N-dimethylglycine, and valine in diagnostics of renal function. Thus, if renal function is to be diagnosed on a patient, such a marker set is very well suited. The diagnosis of renal function can encompass, e.g., revealing an impaired GFR and/or a pathophysiologic factor of such an impaired GFR.

In an aspect, the proposed solution relates to an in vitro method for calculating a glomerular filtration rate (GFR) of an individual. Thereby, this method comprises the following steps. First, a blood sample of an individual is provided. Then, this blood sample is analyzed with an appropriate measuring technique to determine the concentration of at least three substances chosen from the group consisting of alanine, choline, creatine, creatinine, dimethyl sulfone, dimethylamine, glucose, glycerol, isoleucine, leucine, myo-inositol, N,N-dimethylglycine, and valine.

Afterwards, a predicted (or estimated) GFR is calculated from the determined concentrations of the substances.

Appropriate measuring techniques are NMR spectroscopy, mass spectrometry, electron spin resonance, vibrational spectroscopy, UV/VIS spectroscopy, fluorescence spectroscopy and X-ray spectroscopy.

NMR spectroscopy is particularly well suited as measuring techniques. It is possible to work over a very broad measurement range by NMR spectroscopy, namely over more than six orders of magnitude (concentration of the substance in the analysis sample approximately 1 μmold to 1 mol/l). Further, hundreds of substances and substance concentrations are detected in parallel in only a single measurement. Thereby, the accuracy is better than 1% over the whole measurement range. It is particularly favourable that also unexpected ingredients of the sample are detected so that it is not necessary to know already prior to the analysis which substances one wants to detect. In case of a human blood sample, it can be drawn upon more than 1500 single signals by an NMR spectroscopic analysis, which signals in each case represent single, pure, quantifiable conditions.

Mass spectrometry is likewise particularly well suited. Mass spectrometry is a highly sensitive measuring technique. Like in case of NMR spectroscopy, the signal intensities obtained by mass spectrometry correspond to the concentration of a substance. The position of the signals indicates both in the case of NMR spectroscopy as well as in the case of mass spectrometry the nature of the substance, even if the physical basis of both spectroscopic methods is different. Thus, the evaluation proceedings are very similar in case of both methods.

In an embodiment, the term “blood” comprises full blood, blood serum or blood plasma. In an embodiment, the term “blood” denotes blood serum. In an embodiment, the term “blood” denotes blood plasma.

To set up a statistical model that predicts the GFR for given substance integrals, many regression approaches are thinkable. To keep the model simple, and as more complex modelling approaches did not show superiority, linear regression modelling was selected in an embodiment. Second-order interactions were included, as some interactions between individual biomarkers are known from literature, e.g., creatine is a precursor molecule of creatinine. No higher-order interactions were considered to minimize risk of over-fitting.

In an embodiment in which 11 biomarkers of the substances listed above are included, the regression model takes the following form:

$${GFR}_{novel biomarkers} = {β}_{0} + {\sum }_{i = 1}^{11} {β}_{i} {x}_{i} + {\sum }_{j = 1}^{10} {\sum }_{k = j + 1}^{11} {β}_{jk} {x}_{j} {x}_{k} + ɛ , \qquad Equation 1$$

wherein xᵢ represents each of the 11 substance integrals, βᵢ represents the corresponding model coefficients, β₀ represents the intercept, and ε denotes an error term. If more or less biomarkers are used, the indices i, j, and k are adjusted accordingly.

In an embodiment, the GFR range was divided into only two sections for local modelling. As transition area, the region between 60 and 90 ml/min/1.73 m² was chosen as this choice resulted in the most robust results. In addition, the area is known as “creatinine blind spot” and thus most difficult to model.

In an embodiment, the two local model predictions were combined. A straight forward interpolation function was applied that combined the two prediction values of the local models. This approach made use of weighting the models depending on the distance to the GFR limits 0 and 150, of being continuous, and of being flexible due to a parameter p:

$$\begin{matrix}{GFR}_{combined} = \{ \begin{matrix}{\hat{y}}_{A} \\ {\hat{y}}_{B} \\ \frac{{( 150 - {\hat{y}}_{B} )}^{p}}{{{\hat{y}}_{A}}^{p} + {( 150 - {\hat{y}}_{B} )}^{p}} {\hat{y}}_{A} + \frac{{\hat{y}}_{A}}{{{\hat{y}}_{A}}^{p} + {( 150 - {\hat{y}}_{B} )}^{p}} {\hat{y}}_{B} \end{matrix} & Equation 2 \\ \begin{matrix}{\hat{y}}_{A} < 0 , {\hat{y}}_{B} \leq 150 , \\ {\hat{y}}_{B} > 150 , \\ else .\end{matrix} & \end{matrix}$$

\hat{γ}_{A} and \hat{γ}_{B} are the prediction values of the lower and the upper model, respectively, and p is a power parameter that can be in range of 1 to 10. In a comparison study, it was shown that a value of p=4 was a suitable choice.

In an embodiment, the predicted GFR is calculated by the following equation:

GFR=a−b log(substance 1)−c log(substance 2)*d log(substance 3) Equation 3

### Thereby,

a is a number in the range of 100 to 260, in particular of 110 to 250, in particular of 120 to 240, in particular of 130 to 230, in particular of 140 to 220, in particular of 150 to 210, in particular of 160 to 200, in particular of 170 to 190, in particular of 180 to 190;
b is a number in the range of 10 to 100, in particular of 20 to 90, in particular of 30 to 80, in particular of 40 to 70, in particular of 50 to 60;
c is a number in the range of 1 to 10, in particular of 2 to 19, in particular of 3 to 18, in particular of 4 to 17, in particular of 5 to 16, in particular of 6 to 15, in particular of 7 to 14, in particular of 8 to 13, in particular of 9 to 12, in particular of 10 to 11;
d is a number in the range of 10 to 100, in particular of 20 to 90, in particular of 30 to 80, in particular of 40 to 70, in particular of 50 to 60; and

(substance n) denotes the concentration of the respective substance n.

This equation is particularly appropriate to describe a global linear regression model over a GFR range from 0 to 150 mL/min/1.73 m².

In an embodiment, substance 1 is creatinine, substance 2 is dimethyl sulfone, and substance 3 is valine. In an embodiment, substance 1 is creatinine, substance 2 is myo-inositol, and substance 3 is valine. In an embodiment, substance 1 is isoleucine, substance 2 is dimethylamine, and substance 3 is valine. In an embodiment, substance 1 is leucine, substance 2 is N,N-dimethylglycine, and substance 3 is creatine. In an embodiment, substance 1 is creatinine, substance 2 is glycerol, and substance 3 is dimethyl sulfone.

In an embodiment, the predicted GFR is calculated by the following equation:

GFR=exp(f)*(substance 4)^{-g}* (substance 5)⁻*substance 6)ⁱ Equation 4

Thereby,
f is a number in the range of 1 to 20, in particular of 2 to 19, in particular of 3 to 18, in particular of 4 to 17, in particular of 5 to 16, in particular of 6 to 15, in particular of 7 to 14, in particular of 8 to 13, in particular of 9 to 12, in particular of 10 to 11;
g is a number in the range of 0.1 to 2.0, in particular of 0.2 to 1.9, in particular of 0.3 to 1.8, in particular of 0.4 to 1.7, in particular of 0.5 to 1.6, in particular of 0.6 to 1.5, in particular of 0.7 to 1.4, in particular of 0.8 to 1.3, in particular of 0.9 to 1.2, in particular of 1.0 to 1.1;
h is a number in the range of 0.1 to 2.0, in particular of 0.2 to 1.9, in particular of 0.3 to 1.8, in particular of 0.4 to 1.7, in particular of 0.5 to 1.6, in particular of 0.6 to 1.5, in particular of 0.7 to 1.4, in particular of 0.8 to 1.3, in particular of 0.9 to 1.2, in particular of 1.0 to 1.1;
i is a number in the range of 0.1 to 2.0, in particular of 0.2 to 1.9, in particular of 0.3 to 1.8, in particular of 0.4 to 1.7, in particular of 0.5 to 1.6, in particular of 0.6 to 1.5, in particular of 0.7 to 1.4, in particular of 0.8 to 1.3, in particular of 0.9 to 1.2, in particular of 1.0 to 1.1; and

(substance n) denotes the concentration of the respective substance n.

In an embodiment, substance 4 is creatinine, substance 5 is myo-inositol, and substance 6 is valine. In an embodiment, substance 4 is creatine, substance 5 is glycerol, and substance 6 is leucine. In an embodiment, substance 4 is dimethylamine, substance 5 is isoleucine, and substance 6 is dimethyl sulfone. In an embodiment, substance 4 is creatinine, substance 5 is glycerol, and substance 6 is isoleucine.

This equation is particularly appropriate to describe a global log-log regression model over a GFR range from 0 to 150 mL/min/1.73 m².

In an embodiment, the predicted GFR is calculated by the following equations:

GFR_{A}=exp(−j)*(substance 7)^{-k}*(substance 8)^{l}*(substance 9)^{m}*(substance 7)^{o log}(substance 8), Equation 5

GFR_{B}=p−q log(substance 7)−r log(substance 10) Equation 6

Thereby,
j is a number in the range of 0.1 to 10, in particular of 0.5 to 9, in particular of 1.0 to 8, in particular of 1.5 to 7, in particular of 2.0 to 6, in particular of 2.5 to 5, in particular of 3.0 to 4;
k is a number in the range of 0.1 to 2.0, in particular of 0.2 to 1.9, in particular of 0.3 to 1.8, in particular of 0.4 to 1.7, in particular of 0.5 to 1.6, in particular of 0.6 to 1.5, in particular of 0.7 to 1.4, in particular of 0.8 to 1.3, in particular of 0.9 to 1.2, in particular of 1.0 to 1.1;
l is a number in the range of 0.1 to 10, in particular of 0.5 to 9, in particular of 1.0 to 8, in particular of 1.5 to 7, in particular of 2.0 to 6, in particular of 2.5 to 5, in particular of 3.0 to 4;
m is a number in the range of 0.1 to 2.0, in particular of 0.2 to 1.9, in particular of 0.3 to 1.8, in particular of 0.4 to 1.7, in particular of 0.5 to 1.6, in particular of 0.6 to 1.5, in particular of 0.7 to 1.4, in particular of 0.8 to 1.3, in particular of 0.9 to 1.2, in particular of 1.0 to 1.1;
o is a number in the range of 0.1 to 2.0, in particular of 0.2 to 1.9, in particular of 0.3 to 1.8, in particular of 0.4 to 1.7, in particular of 0.5 to 1.6, in particular of 0.6 to 1.5, in particular of 0.7 to 1.4, in particular of 0.8 to 1.3, in particular of 0.9 to 1.2, in particular of 1.0 to 1.1;
p is a number in the range of 100 to 360, in particular of 110 to 350, in particular of 120 to 340, in particular of 130 to 330, in particular of 140 to 320, in particular of 150 to 310, in particular of 160 to 300, in particular of 170 to 290, in particular of 180 to 280, in particular of 190 to 270, in particular of 200 to 260, in particular of 210 to 250, in particular of 220 to 240,
q is a number in the range of 10 to 100, in particular of 20 to 90, in particular of 30 to 80, in particular of 40 to 70, in particular of 50 to 60;
r is a number in the range of 1 to 40, in particular of 2 to 35, in particular of 3 to 30, in particular of 4 to 25, in particular of 5 to 20, in particular of 6 to 19, in particular of 7 to 18, in particular of 8 to 17, in particular of 9 to 16, in particular of 10 to 15, in particular of 11 to 14, in particular of 12 to 13;

(substance n) denotes the concentration of the respective substance n;

GFR_{A} denotes the predicted GFR for a first GFR range and

GFR_{B} denotes the predicted GFR for a second GFR range.

In an embodiment, the first GFR range encompasses GFRs indicative for hypofiltration and the second GFR range encompasses GFRs of indicative for hyperfiltration. To give an example, the first GFR range encompasses in an embodiment GFRs of 0 mL/min/1.73 m² to 90 mL/min/1.73 m², and the second GFR range encompasses GFRs of 60 mL/min/1.73 m² to 150 mL/min/1.73 m².

In an embodiment, substance 7 is creatinine, substance 8 is myo-inositol, substance 9 is valine, and substance 10 is dimethyl sulfone. In an embodiment, substance 7 is creatine, substance 8 is myo-inositol, substance 9 is leucine, and substance 10 is dimethyl sulfone. In an embodiment, substance 7 is glycerol, substance 8 is N,N-dimethylglycine, substance 9 is alanine, and substance 10 is dimethylamine. In an embodiment, substance 7 is choline, substance 8 is glucose, substance 9 is isoleucine, and substance 10 is dimethylamine. In an embodiment, substance 7 is creatinine, substance 8 is dimethyl sulf one, substance 9 is valine, and substance 10 is dimethylamine.

This equation is particularly appropriate to describe a local model, wherein the equations for describing the lower part of the whole GFR range (GFR_{A}) and the upper part of the whole GFR range (GFRB) can be optimized prior to combining them.

In an embodiment, the predicted GFR is calculated by the following equations:

GFR_{A}=exp(s)*(substance 11)^{t}*(substance 12)^{u}* (substance 11)^{-v}log(substance 13), Equation 7

GFR_{B}=w−x log(substance 14)−ylog(substance 15)−zlog(substance 14)*log(substance 13) Equation 8

Thereby,
s is a number in the range of 0.1 to 10, in particular of 0.5 to 9, in particular of 1.0 to 8, in particular of 1.5 to 7, in particular of 2.0 to 6, in particular of 2.5 to 5, in particular of 3.0 to 4;
t is a number in the range of 0.1 to 10, in particular of 0.5 to 9, in particular of 1.0 to 8, in particular of 1.5 to 7, in particular of 2.0 to 6, in particular of 2.5 to 5, in particular of 3.0 to 4;
u is a number in the range of 0.1 to 2.0, in particular of 0.2 to 1.9, in particular of 0.3 to 1.8, in particular of 0.4 to 1.7, in particular of 0.5 to 1.6, in particular of 0.6 to 1.5, in particular of 0.7 to 1.4, in particular of 0.8 to 1.3, in particular of 0.9 to 1.2, in particular of 1.0 to 1.1;
v is a number in the range of 0.1 to 2.0, in particular of 0.2 to 1.9, in particular of 0.3 to 1.8, in particular of 0.4 to 1.7, in particular of 0.5 to 1.6, in particular of 0.6 to 1.5, in particular of 0.7 to 1.4, in particular of 0.8 to 1.3, in particular of 0.9 to 1.2, in particular of 1.0 to 1.1;
w is a number in the range of 50 to 220, in particular of 60 to 210, in particular of 70 to 200, in particular of 80 to 190, in particular of 90 to 180, in particular of 100 to 170, in particular of 110 to 160, in particular of 120 to 150, in particular of 130 to 140;
x is a number in the range of 10 to 100, in particular of 20 to 90, in particular of 30 to 80, in particular of 40 to 70, in particular of 50 to 60;
y is a number in the range of 1 to 40, in particular of 2 to 35, in particular of 3 to 30, in particular of 4 to 25, in particular of 5 to 20, in particular of 6 to 19, in particular of 7 to 18, in particular of 8 to 17, in particular of 9 to 16, in particular of 10 to 15, in particular of 11 to 14, in particular of 12 to 13;
z is a number in the range of 1 to 40, in particular of 2 to 35, in particular of 3 to 30, in particular of 4 to 25, in particular of 5 to 20, in particular of 6 to 19, in particular of 7 to 18, in particular of 8 to 17, in particular of 9 to 16, in particular of 10 to 15, in particular of 11 to 14, in particular of 12 to 13;

(substance n) denotes the concentration of the respective substance n;

GFR_{A} denotes the predicted GFR for a first GFR range and

GFR_{B} denotes the predicted GFR for a second GFR range.

In an embodiment, the first GFR range encompasses GFRs indicative for hypofiltration and the second GFR range encompasses GFRs of indicative for hyperfiltration. To give an example, the first GFR range encompasses in an embodiment GFRs of 0 mL/min/1.73 m² to 90 mL/min/1.73 m², and the second GFR range encompasses GFRs of 60 mL/min/1.73 m² to 150 mL/min/1.73 m².

In an embodiment, substance 11 is dimethyl sulfone, substance 12 is valine, substance 13 is creatinine, substance 14 is creatine, and substance 15 is glycerol. In an embodiment, substance 11 is dimethylamine, substance 12 is leucine, substance 13 is creatinine, substance 14 is glucose, and substance 15 is glycerol. In an embodiment, substance 11 is N,N-dimethylglycine, substance 12 is isoleucine, substance 13 is creatine, substance 14 is choline, and substance 15 is dimethylamine. In an embodiment, substance 11 is myo-inositol, substance 12 is isoleucine, substance 13 is creatinine, substance 14 is creatine, and substance 15 is choline.

This equation is particularly appropriate to describe a local model, wherein the equations for describing the lower part of the whole GFR range (GFR_{A}) and the upper part of the whole GFR range (GFR_{B}) can be optimized prior to combining them.

Instead of using the logarithm to the base of 10, a logarithm to another base, such as the natural logarithm, could be likewise used in any of the before-mentioned equations.

In an embodiment, it is possible to multiply concentrations of different markers with each other. In doing so, a biologic interaction between these markers can be particularly appropriate mapped in the used equation.

In an embodiment, at least a first equation and second equation are combined for calculating the predicted GFR. Thereby, the first equation is optimized for a first GFR range and the second equation is optimized for a second GFR range. Thereby, the first GFR range and the second GFR range are spaced apart from each other, adjoin or partially overlap. If exactly two equations are used for calculating the predicted GFR, the GFR ranges for which these equations are optimized, typically adjoin or partially overlap. A gap between the first GFR range and the second GFR arranged is typically only present, if at least one additional equation is used together with the first equation and the second equation to calculate the predicted GFR. Then, this at least one other equation can be optimized for another GFR range that fills the gap between the first GFR range and the second GFR range.

In an embodiment, the first equation describes or is representative for a first metabolic process and can thus be denoted as first metabolic process model, in particular as first renal metabolic process model. Furthermore, the second equation describes or is representative for a second metabolic process and can thus be denoted as second metabolic process model, in particular as second renal metabolic process model. By combining the first metabolic process model and the second metabolic process model and optionally a further (renal) metabolic process model, a composite organ function model results that combines the properties of the combined metabolic process models in a synergistic way. Such a composite organ function model (being based on at least two equations) has a much better performance than isolated equations trying to describe a complete metabolic space used according to prior art for calculating or estimating the GFR.

In an embodiment, the first GFR range comprises GFRs being indicative for hypofiltration. At the same time, the second GFR range comprises GFRs being indicative for hyperfiltration of a normal glomerular filtration. A typical normal GFR lies in a range between 90 mL/min/1.73 m² (the GFR is regularly standardized to a standard body surface of 1.73 m²) and 120 mL/min/1.73 m² in case of adults being 65 years or younger and between 60 mL/min/1.73 m² and 120 mL/min/1.73 m² in case of adults being elder than 65 years. Typically GFRs in case of hyperfiltration are GFRs of 120 mL/min/1.73 m² or more, in particular 130 mL/min/1.73 m² or more, in particular 140 mL/min/1.73 m² or more, in particular 150 mL/min/1.73 m² or more, or lying in any interval that can be built up from the GFR values.

Typical GFRs indicative for hypofiltration are, in case of adults being 65 years or younger, GFRs of 89 mL/min/1.73 m² and below, in particular GFRs in a range of 0 mL/min/1.73 m² to 89 mL/min/1.73 m², in particular GFRs in a range of 5 mL/min/1.73 m² to 80 mL/min/1.73 m², in particular GFRs in a range of 10 mL/min/1.73 m² to 70 mL/min/1.73 m², in particular GFRs in a range of 15 mL/min/1.73 m² to 60 mL/min/1.73 m², in particular GFRs in a range of 20 mL/min/1.73 m² to 50 mL/min/1.73 m², in particular GFRs in a range of 30 mL/min/1.73 m² to 40 mL/min/1.73 m².

Typical GFRs indicative for hypofiltration are, in case of adults being elder than 65 years, GFRs of 59 mL/min/1.73 m² and below, in particular GFRs in a range of 0 mL/min/1.73 m² to 59 mL/min/1.73 m², in particular GFRs in a range of 5 mL/min/1.73 m² to 55 mL/min/1.73 m², in particular GFRs in a range of 10 mL/min/1.73 m² to 50 mL/min/1.73 m², in particular GFRs in a range of 15 mL/min/1.73 m² to 40 mL/min/1.73 m², in particular GFRs in a range of 20 mL/min/1.73 m² to 30 mL/min/1.73 m².

Appropriate GFRs for the first GFR range are GFRs lying in a range of 0 mL/min/1.73 m² to 89 mL/min/1.73 m², in particular of 5 mL/min/1.73 m² to 80 mL/min/1.73 m², in particular of 10 mL/min/1.73 m² to 70 mL/min/1.73 m², in particular of 15 mL/min/1.73 m² to 60 mL/min/1.73 m², in particular of 15 mL/min/1.73 m² to 59 mL/min/1.73 m², in particular of 20 mL/min/1.73 m² to 50 mL/min/1.73 m², in particular of 30 mL/min/1.73 m² to 40 mL/min/1.73 m².

Appropriate GFRs for the second GFR range GFRs lie in a range of 60 to 150 mL/min/1.73 m², in particular of 65 mL/min/1.73 m² to 140 mL/min/1.73 m², in particular of 70 mL/min/1.73 m² to 130 mL/min/1.73 m², in particular of 80 mL/min/1.73 m² to 120 mL/min/1.73 m², in particular of 90 mL/min/1.73 m² to 110 mL/min/1.73 m², in particular of 95 mL/min/1.73 m² to 105 mL/min/1.73 m².

In an embodiment, the first equation is weighted by a first weighing factor and the second equation is weighted by a second weighing factor. Thereby, the first weighing factor is bigger than the second weighing factor if the predicted GFR falls within the first GFR range but not within the second GFR range. In assessing whether the predicted GFR falls within the first GFR range (but not within the second GFR range), the first equation and/or the second equation are used in a non-weighted form. By applying such weighing factors, it is possible to increase the importance of the first equation in a combination of the first equation and the second equation. Since the first equation is optimized for the first GFR range and, in this embodiment, the predicted GFR falls within the first GFR range, higher weighing of the first equation has the effect that the overall calculation of the GFR is more exact than in case that both the first equation and the second equation were weighted equally.

The first weighing factor is, however, smaller than the second weighing factor if the predicted GFR falls within the second GFR range but not within the first GFR range. Once again, the first equation and the second equation are used in a non-weighted form in order to assess whether or not the predicted GFR falls within the first GFR range or within the second GFR range. If this assessment results in the finding that the predicted GFR falls within the second GFR range, the second equation is higher weighted than the first equation. Like in the preceding explained example, this increases the accuracy of the calculation of the GFR by a combination of the first equation and the second equation since the second equation is optimized for the second GFR range.

If the calculated GFR falls in an overlapping region of the first and the second GFR range, a more detailed assessment is necessary. In an embodiment, a first distance of the calculated GFR to the end of the first GFR range is calculated. Likewise, a second distance between the calculated

GFR and the end of the second GFR range is calculated. The term “end” denotes in each case that end of the respective range that is located in the overlapping region. If the first distance is bigger than the second distance, then the calculated GFR is closer to the first GFR range than to the second GFR range. Therefore, it “belongs” more to the first GFR range than to the second GFR range. Consequently, the first range is weighted stronger than the second. However, the more remote the calculated GFR is located from the center of the respective GFR range the closer it is located to the respective other range. Hence, the smaller is the given predominance of the respective weighing factor over the respective other weighing factor.

In an embodiment, a third equation is combined with the first equation and the second equation to calculate the predicted GFR. Thereby, the third equation is optimized for a third GFR range. The first GFR range, the second GFR range and the third GFR range can be spaced apart from each other, can adjoin can partially overlap with at least one of the respective other GFR ranges. As already explained above, typically each GFR value lying within the whole GFR range to be examined is covered by at least one of these GFR ranges. Thus, if exactly three equations are combined for calculating the predicted GFR, the first GFR range might be spaced apart from the second GFR range, and the third GFR range might partially overlap with both the first GFR range and the second GFR range so as to fully cover the gap between the first GFR range and the second GFR range.

It is possible to use more than three equations. Then, the individual GFR ranges assigned to each equation are spaced apart from each other, adjoin or partially overlap with at least one of the respective other GFR ranges, wherein, in an embodiment, each GFR value lying within the whole GFR range to be examined is covered by at least one GFR range.

In an embodiment, a classification of the individual, the blood sample of whom was analyzed, into a predefined class or group is done. This classification takes place based on a pattern of the determined concentrations of the substances.

To give an example, if the concentration of substances A and B is high and the concentration of substance C is low (and the calculated GFR is lower or higher than a normal GFR), then this individual is classified into a first group. If the concentration of substance A is high, the concentration of substance B is low and the concentration of substance C is high (and the calculated GFR is higher or lower than a normal GFR), then this individual is classified into second group. In doing so, a plurality of groups or classes can be defined and the individuals, the blood samples of whom have been analyzed, can be classified into these groups or classes. Then, individuals having a similar pathophysiologic ground for an impaired GFR are grouped together.

In an embodiment, a pathophysiologic classification of the individual, the blood sample of whom was analyzed, into a predefined pathophysiological class or group is done. This classification takes place based on a pattern of the determined concentrations of the substances. In an embodiment, the pathophysiological group is defined by at least one conspicuous pathophysiologic factor chosen from the group consisting of renal filtration, reabsorption and/or secretion capability, uremia, renal metabolic acidosis, renal oxidative stress, altered renal gluconeogenesis, amyotrophia, sarcopenia, changes of gut microbiome and/or tubular hyperosmolality.

In an embodiment, the value of the calculated GFR can also be used to define the (pathophysiological) groups and to classify the individual into one of the predefined (pathophysiological) groups.

It is possible to define a certain therapy for each (pathophysiological) group so that an individual, who is grouped into a specific (pathophysiological) group, can undergo the respective therapy assigned to this group.

Some of the individuals to be classified into one of the different groups can be undoubtedly classified into a specific group. In case of other individuals, such a classification might be more complicated. To be more precise, some individuals might be classified into a first defined group or a second predefined group. Then, quantitative data can be used to assign these individuals to, e.g., the first group and indicate that they have a tendency to be in transition into the second (or another) group. This tendency exists, in this example, because the considered markers have a concentration pattern that is closely related to the concentration pattern of the second group. Therefore, in an embodiment, a likelihood can be indicated that the classified individual is in transition to another predefined class. Thus, the performed classification can, in this embodiment, denote “first group with a strong tendency to second group”. In such a case, a combination of the therapies assigned to the first group and to the second group can be applied to the respective individual in order to ameliorate his or her renal state.

In an embodiment, the method does not stop at simply indicating a precisely calculated GFR, but also gives information on the pathophysiologic background of the individual for whom the GFR is calculated. If the calculated GFR is indicative of hypofiltration or hyperfiltration, the determined concentrations of the substances are, in this embodiment, additionally used to provide an indicator of a pathophysiologic factor of an impaired GFR. In doing so, the determined concentration of at least one of the substances is compared with a concentration of the same substance in the blood of another individual having a similar GFR. Thereby, no 1:1 comparison needs to be made. Rather, the comparison can be done with respect to one or more previously obtained concentration values that can optionally be stored in a database. Likewise, the term “concentration of the same substance in the blood of another individual having a similar GFR” also encompasses an average value of a plurality of such concentration values of different individuals having similar GFRs.

By such a comparison, conclusions can be drawn between different individuals, whether the pathophysiologic background of the observed impaired GFR is the same or different. This information is very helpful for future decisions on therapeutic treatments, since the GFR itself only indicates that the individual has an improper renal function. Thus, the GFR merely indicates that there is something wrong with the renal function. An insight into the pathophysiologic background by additionally providing an indicator of a pathophysiologic factor of the impaired GFR addresses the question, why there is something wrong with the renal function.

In an embodiment, the pathophysiologic factor determined by the novel biomarkers is chosen from the categories consisting of renal failure, renal co-morbidity, and extra-renal co-morbidity. Thereby, it is possible to determine pathophysiologic factors belonging to different categories.

Specific examples of the pathophysiologic factor determined in an embodiment are renal filtration, reabsorption and/or secretion capability, uremia, renal metabolic acidosis, renal oxidative stress, altered renal gluconeogenesis, amyotrophia, creatinine to muscle mass ratio, changes of gut microbiome and/or tubular hyperosmolality. Thereby, it is possible to determine a single or more than one of these pathophysiologic factors, wherein any combination of the pathophysiologic factors is possible.

In an embodiment, the indicator of the pathophysiologic factor of an impaired GFR and optionally also the predicted GFR itself serves as basis for at least one further action chosen from the group consisting of assisting in assessing the eligibility of diabetic patients for metformin therapy; assisting in evaluating a contribution of kidney dysfunction to edema formation for congestive heart failure patients; supporting a diagnosis of kidney disease in children; assisting in identifying suitable candidates for live kidney donation; assisting in evaluating kidney function after renal allograft transplantation; supporting an evaluation of the eligibility of chronic kidney disease stage IV patients to enter kidney transplant waiting list; assisting in evaluating the risk for acute kidney injury peri liver transplantation and/or post liver transplantation; assisting GFR estimation in patients in patients having a body constitution significantly differing from the body constitution of a healthy standard population (i.e., patients with extreme body composition), e.g., patients suffering from obesity, sarcopenia, frailty or amputations; assisting in evaluating the necessity of liver and kidney co-transplantation; and assisting in adapting a drug dose administered to a patient according to the renal function of the patient, e.g. a dose of antibiotics like aminoglycosides, metronidazole and/or vancomycin.

These assisting or supporting steps in other methods do not directly relate to a therapeutic or diagnostic procedure. They constitute just further pieces of the puzzle given a physician at hand to make him subsequent decisions easier.

In an aspect, the proposed solution relates to a medical method for calculating the GFR of an individual in need thereof, comprising the following steps: First, a blood sample of an individual is obtained. Then, this blood sample is analyzed with an appropriate measuring technique to determine the concentration of at least three substances chosen from the group consisting of alanine, choline, creatine, creatinine, dimethyl sulfone, dimethylamine, glucose, glycerol, isoleucine, leucine, myo-inositol, N,N-dimethylglycine, and valine. Afterwards, a predicted (or estimated) GFR is calculated from the determined concentrations of the substances.

In an embodiment of this medical method, information on the pathophysiologic background of the individual for whom the GFR is calculated is additionally obtained. If the calculated GFR is indicative of hypofiltration or hyperfiltration, the determined concentrations of the substances are, in this embodiment, additionally used to provide an indicator of a pathophysiologic factor of an impaired GFR. In doing so, the determined concentration of at least one of the substances is compared with a concentration of the same substance in the blood of another individual having a similar GFR. Reference is made to the detailed explanations in this circumstance with respect to the in vitro method.

In an embodiment, the method comprises the step of outputting obtained/calculated data in form of a digital and/or printed report to the individual and/or to medical staff. Such a report comprises, in an embodiment, the calculated value of the GFR estimated by using the novel biomarkers. In an embodiment, the report additionally comprises the concentrations of the measured substances. Thereby, it can represent the concentrations on a scale indicating appropriate upper and lower thresholds of concentrations of the respective substances. Then, it can be easily assessed, whether or not the determined concentrations are within or outside the specified concentration range. In an embodiment, the report comprises information on the pathophysiologic background related to the determined concentrations. To give an example, the report can state that an increased serum level of substance A is associated with a certain pathophysiologic state, e.g., with metabolic acidosis or oxidative stress. In an embodiment, the report comprises citations of scientific literature to supplement the pathophysiologic background. In an embodiment, the pathophysiologic background on the report relates only to that substances of which increased/decreased serum levels of the respective substances have been found in the examined sample. Thus, in this embodiment, the report only presents information to the individual and/or the medical staff (like a physician examining the individual) that is in fact relevant for the individual based on the determined concentrations of the analyzed blood sample of the individual.

In an aspect, the proposed solution relates to medical method aiming at one of the following aspects by using the indicator of a pathophysiologic factor of an impaired GFR: deciding on the eligibility of diabetic patients for metformin therapy; evaluating a contribution of kidney dysfunction to edema formation for congestive heart failure patients; diagnosing kidney disease in children; identifying suitable candidates for live kidney donation; evaluating kidney function after renal allograft transplantation; deciding on the eligibility of chronic kidney disease stage IV patients to enter kidney transplant waiting list; evaluating the risk for acute kidney injury peri liver transplantation and/or post liver transplantation; GFR estimation in patients having a body constitution significantly differing from the body constitution of a healthy standard population (i.e., patients with extreme body composition), e.g., patients suffering from obesity, sarcopenia, frailty or amputations; deciding on the necessity of liver and kidney co-transplantation; and adapting a drug dose administered to a patient according to the renal function of the patient, e.g. a dose of antibiotics like aminoglycosides, metronidazole and/or vancomycin.

All aspects and embodiments of the described uses and the described methods can be combined in any desired way and can be transferred from the uses to the methods and vice versa as well as between different uses and different methods in any desired way.

### BRIEF DESCRIPTION OF THE DRAWINGS

Further details of aspects of the solution will be explained in the following with respect to exemplary embodiments and accompanying Figures.

FIG. 1 shows a correlation plot indicating cross correlations between the individual substances from which the marker set can be built up;

FIG. 2A shows a correlation plot between measured GFR (mGFR) and the GFR estimated by the established CKD-EPI formula (eGFR_{CKD-EPI}) of different patients;

FIG. 2B shows a correlation plot between measured GFR (mGFR) and the GFR estimated by using a first marker set according to an embodiment (eGFR_{novel markes}) of the same patients as in FIG. 2A;

FIG. 2C shows a correlation plot between measured GFR (mGFR) and the GFR estimated by using a second marker set according to an embodiment (eGFR_{novel markes}) of the same patients as in FIG. 2A;

FIG. 2D shows a correlation plot between measured GFR (mGFR) and the GFR estimated by using a third marker set according to an embodiment (eGFR_{novel markes}) of the same patients as in FIG. 2A;

FIG. 2E shows a correlation plot between measured GFR (mGFR) and the GFR estimated by using a forth marker set according to an embodiment (eGFR_{novel markes}) of the same patients as in FIG. 2A;

FIG. 3A shows a correlation plot between measured GFR (mGFR) and the GFR estimated by the established CKD-EPI formula (eGFR_{GKD-EPI}) of different congestive heart failure patients;

FIG. 3B shows a correlation plot between measured GFR (mGFR) and the GFR estimated by using a marker set according to an embodiment (eGFR_{novel markes}) of the same congestive heart failure patients as in FIG. 3A;

FIG. 4A shows a correlation plot between measured GFR (mGFR) and the GFR estimated by the established CKD-EPI formula (eGFR_{GKD-EPI}) of different liver failure patients;

FIG. 4B shows a correlation plot between measured GFR (mGFR) and the GFR estimated by using a marker set according to an embodiment (eGFR_{novel markes}) of the same liver failure patients as in FIG. 4A;

FIG. 5A shows a correlation plot between measured GFR (mGFR) and the GFR estimated by the established CKD-EPI formula (eGFR_{GKD-EPI}) based on cystatin C of different pediatric patients;

FIG. 5B shows a correlation plot between measured GFR (mGFR) and the GFR estimated by the established CKD-EPI formula (eGFR_{GKD-EPI}) based on creatinine of the same pediatric patients as in FIG. 5A;

FIG. 5C shows a correlation plot between measured GFR (mGFR) and the GFR estimated by using a marker set according to an embodiment (eGFR_{novel markes}) of the same pediatric patients as in FIG. 5A;

FIG. 6A shows a renal panel of a first congestive heart failure patient having a GFR of less than 15 mL/min/1.73 m²;

FIG. 6B shows a renal panel of a second congestive heart failure patient having a GFR of less than 15 mL/min/1.73 m²;

FIG. 6C shows a renal panel of a third congestive heart failure patient having a GFR of less than 15 mL/min/1.73 m²; and

FIG. 7 shows an embodiment of a kidney function report.

### DETAILED DESCRIPTION OF THE INVENTION

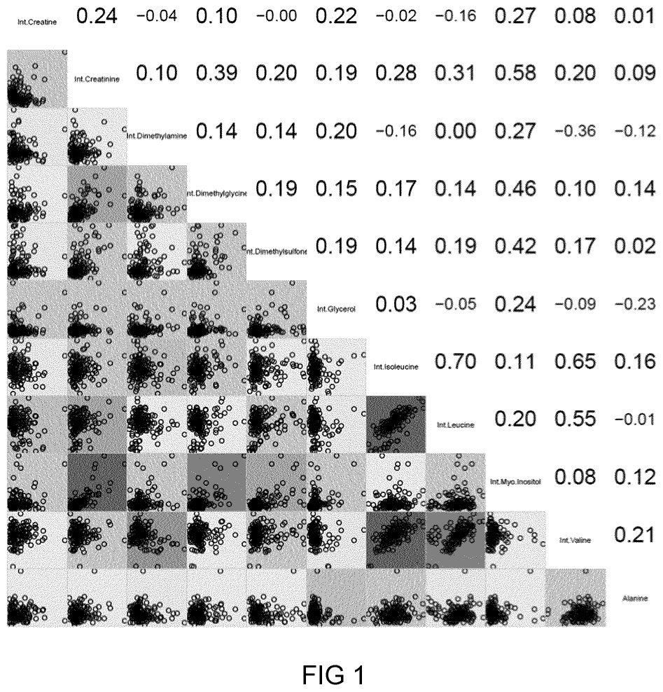

FIG. 1 shows a correlation plot indicating cross correlations between the individual substances of the substance pool that can be used for building up the marker set according to an aspect of the proposed solution. The correlation plot shows in its lower left half correlation panels between individual substances. In its upper right half it shows correlation coefficients of those correlation panels. Thus, each correlation panel in the lower left half corresponds to a correlation coefficient in the upper right half. The lighter grey the correlation panel of two substances, the lower is the correlation between the substances.

This correlation plot was used to elucidate any cross correlations between the substances that go beyond the GFR to be calculated. By this correlation plot, it was possible to show that some of the substances which are present in the substance pool correlate with each other so that they do not well supplement each other for calculating the GFR. Consequently, it is not the first choice to combine two of strongly correlating substances with each other to build up the marker set for calculating the GFR. To give an example, isoleucine and leucine show a strong correlation having a correlation coefficient of 0.70. Thus, both isoleucine and leucine behave very similar with an increasing or decreasing GFR. If both isoleucine and leucine a combined in a marker set for calculating the GFR, one still obtains satisfying results. However, the reliability of a marker set is even higher if less correlating substances are combined with each other. To give an example, and valine and myo-inositol have a correlation coefficient of 0.08, i.e. a very low correlation. Thus, it is more appropriate to combine myo-inositol and valine in a marker set than isoleucine and leucine.

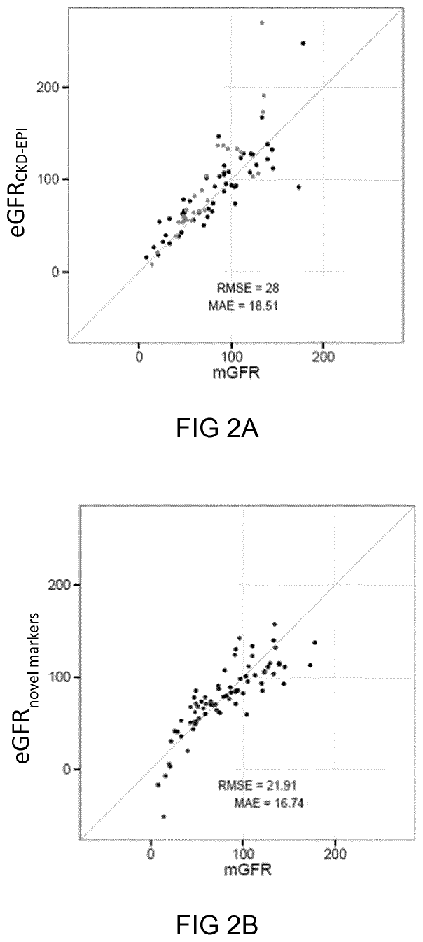

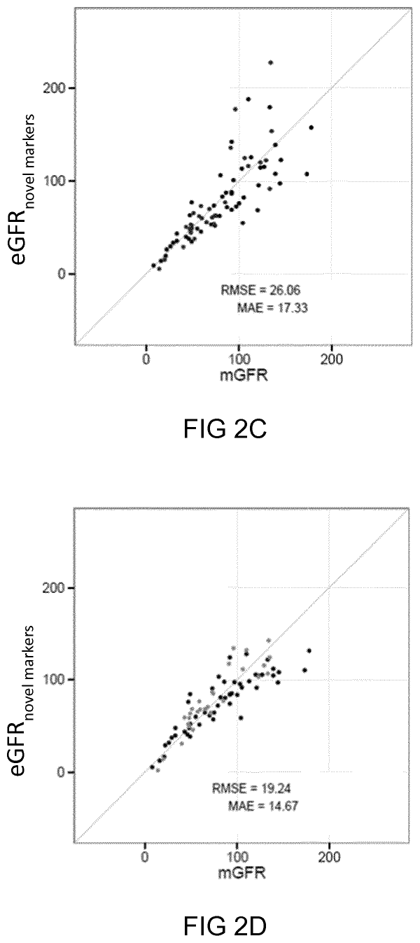

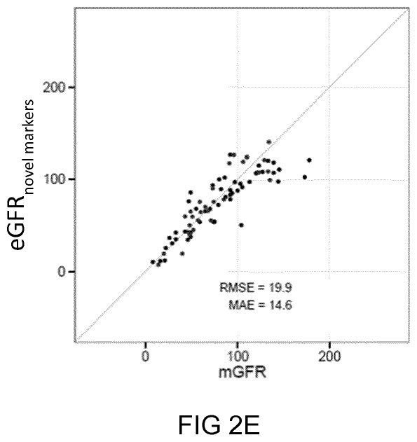

FIG. 2A shows a correlation plot between measured GFR (mGFR) and the GFR estimated by the established CKD-EPI formula or equation (eGFR_{CDK-EPI}) of different patients. The coloring of the data points is of no importance. The overall root mean square error (RMSE) amounts to 28. The mean absolute error (MAE) amounts to 18.51.

FIG. 2B shows a similar correlation plot for the same patients in which the measured GFR and the GFR estimated by using a novel marker set comprising creatinine, dimethyl sulfone, and valine are illustrated. Thereby, equation 3 as explained above has been applied to calculate the GFR. The root mean square error amounts to only 21.91 and the mean absolute error amounts to only 16.74. Thus, the novel biomarkers allow a much more exact and reliable estimation of the GFR than the established methods known from prior art. Expressed in other words, the novel biomarkers allow the calculation of an eGFR that maps the measured GFR much more accurate.

FIG. 2C shows a similar correlation plot for the same patients in which the measured GFR and the GFR estimated by using a novel marker set comprising creatinine, myo-inositol, and valine are illustrated. Thereby, equation 4 as explained above has been applied to calculate the GFR. The root mean square error amounts to only 26.06 and the mean absolute error amounts to only 17.33. Thus, also this novel combination of biomarkers allows a much more exact and reliable estimation of the GFR than the established methods known from prior art.

FIG. 2D shows a similar correlation plot for the same patients in which the measured GFR and the GFR estimated by using a novel marker set comprising creatinine, myo-inositol, valine, and dimethyl sulfone are illustrated. Thereby, equations 5 and 6 as explained above have been applied to calculate the GFR. The root mean square error amounts to only 19.24 and the mean absolute error amounts to only 14.67. Thus, also this novel combination of biomarkers allows a much more exact and reliable estimation of the GFR than the established methods known from prior art.

FIG. 2E shows a similar correlation plot for the same patients in which the measured GFR and the GFR estimated by using a novel marker set comprising creatinine, creatine, dimethyl sulfone, glycerol, and valine are illustrated. Thereby, equations 7 and 8 as explained above have been applied to calculate the GFR. The root mean square error amounts to only 19.9 and the mean absolute error amounts to only 14.6. Thus, also this novel combination of biomarkers allows a much more exact and reliable estimation of the GFR than the established methods known from prior art.

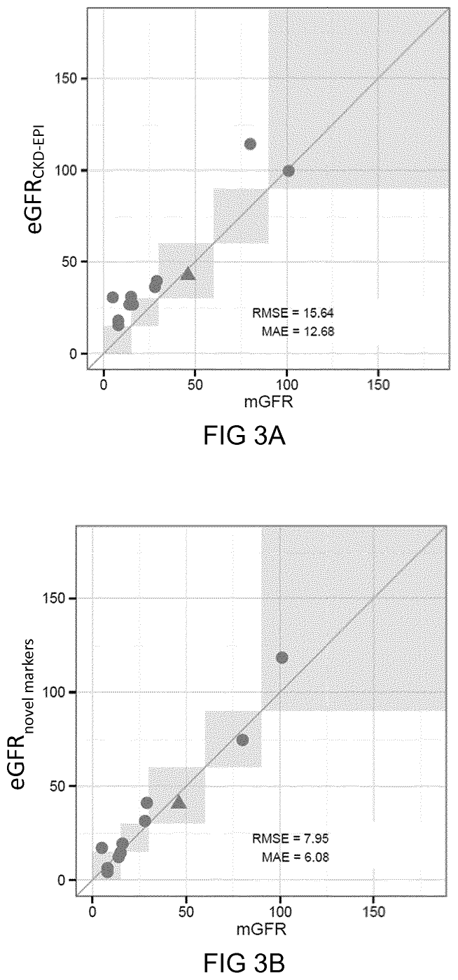

FIG. 3A shows a correlation plot between measured GFR (mGFR) and the GFR estimated by the established CKD-EPI formula or equation (eGFR_{CKD-EPI}) of different patients suffering from congestive heart failure. The overall root mean square error (RMSE) amounts to 15.64. The mean absolute error (MAE) amounts to 12.68. The grey squares in the background indicate five different chronic kidney disease (CKD) stages according to the International Classification of Diseases (ICD) in its 10^{th} revision (ICD-10).

FIG. 3B shows a similar correlation plot for the same patients in which the measured GFR and the GFR estimated by using a novel marker set comprising creatinine, myo-inositol, valine, and dimethyl sulfone are illustrated. Thereby, equations 5 and 6 as explained above have been applied to calculate the GFR. The root mean square error amounts to only 7.95 and the mean absolute error amounts to only 6.08. Thus, the novel biomarkers allow a much more exact and reliable estimation of the GFR than the established methods known from prior art.

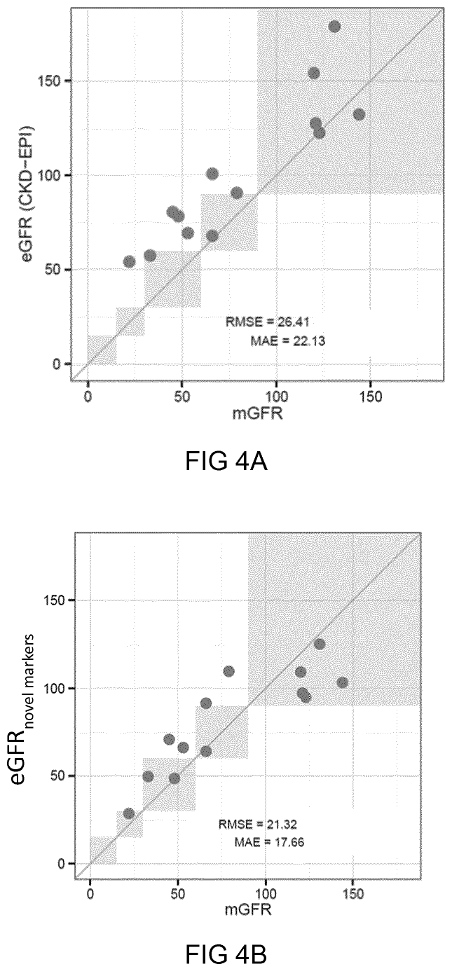

FIG. 4A shows a correlation plot between measured GFR (mGFR) and the GFR estimated by the established CKD-EPI formula (eGFR_{CKD-EPI}) of different patients suffering from liver failure. The overall root mean square error (RMSE) amounts to 26.41. The mean absolute error (MAE) amounts to 22.13.

FIG. 4B shows a similar correlation plot for the same patients in which the measured GFR and the GFR estimated by using a novel marker set comprising creatinine, myo-inositol, valine, and dimethyl sulfone are illustrated. Thereby, equations 5 and 6 as explained above have been applied to calculate the GFR. The root mean square error amounts to only 21.32 and the mean absolute error amounts to only 17.66. Thus, the novel biomarkers allow a much more exact and reliable estimation of the GFR than the established methods known from prior art.

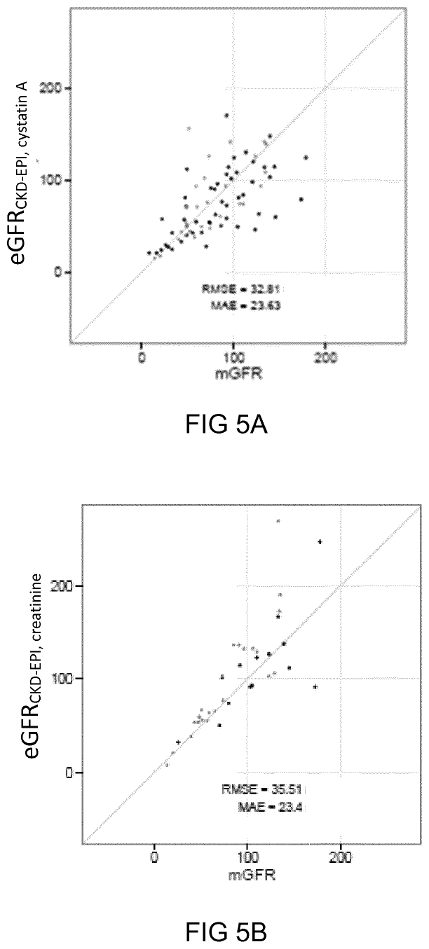

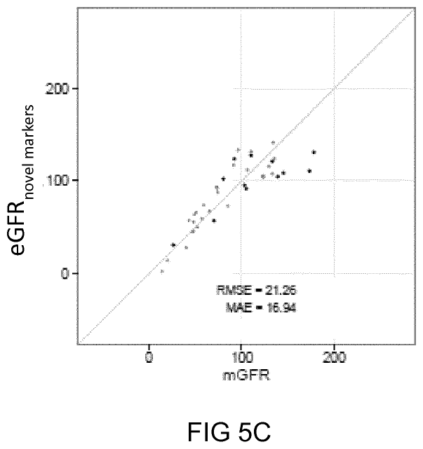

FIG. 5A shows a correlation plot between measured GFR (mGFR) and the GFR estimated by the established CKD-EPI formula or equation (eGFR_{CKD-EPI, cystatin A}) on the basis of cystatin A of different pediatric patients. The coloring of the data points is of no importance. The overall root mean square error (RMSE) amounts to 32.81. The mean absolute error (MAE) amounts to 23.63.

FIG. 5B shows a correlation plot between measured GFR (mGFR) and the GFR estimated by the established CKD-EPI formula or equation (eGFR_{CKD-EPI, creatinine}) on the basis of creatinine of the same pediatric patients. The coloring of the data points is of no importance. The overall root mean square error (RMSE) amounts to 35.51 and is thus worse than in case of using eGFR_{CKD-EPI, cystatin A}. This is in line with the recommendation to calculate the GFR on the basis of cystatin A in case of pediatric patients. The mean absolute error (MAE) amounts to 23.4.

FIG. 5C shows a similar correlation plot for the same pediatric patients in which the measured GFR and the GFR estimated by using a novel marker set comprising creatinine, myo-inositol, valine, and dimethyl sulfone are illustrated. Thereby, equations 5 and 6 as explained above have been applied to calculate the GFR. The root mean square error amounts to only 21.25 and the mean absolute error amounts to only 16.94. Thus, also in case of pediatric patients, this novel combination of biomarkers allows a much more exact and reliable estimation of the GFR than the established methods known from prior art.

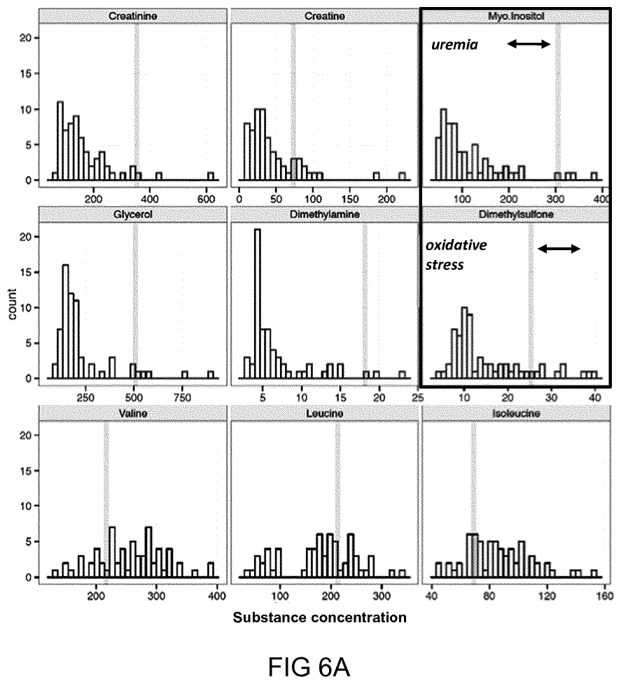

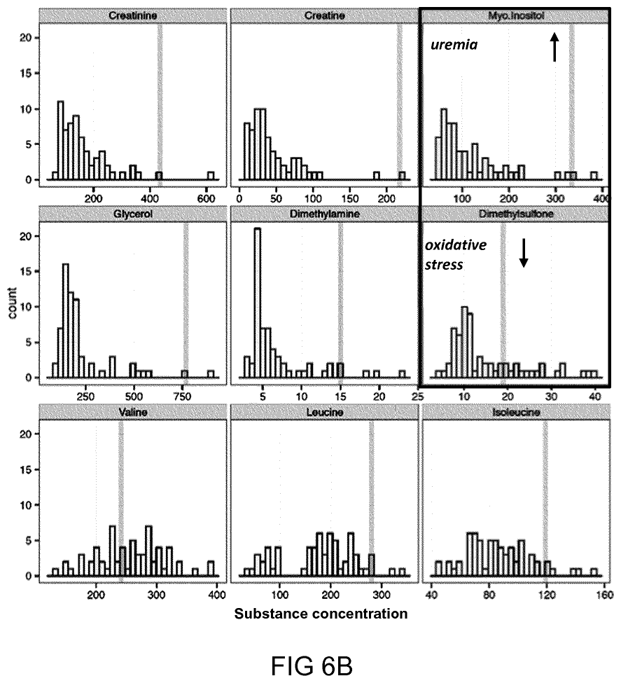

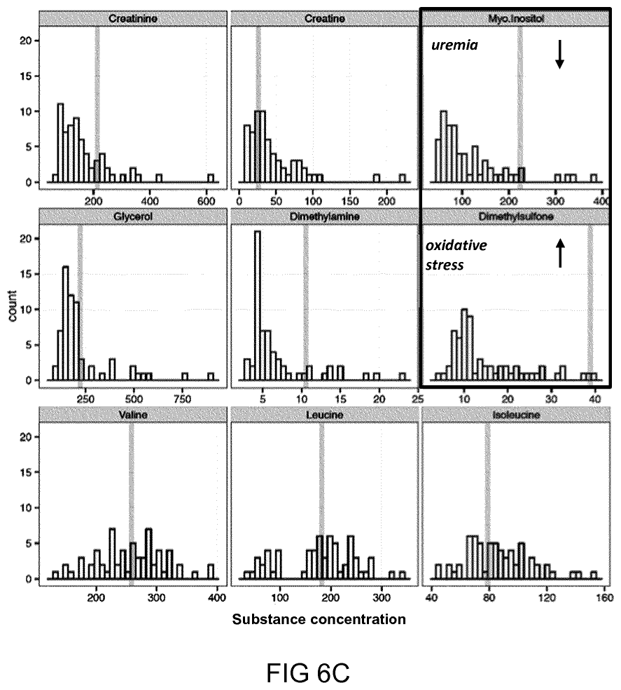

FIGS. 6A, 6B and 6C show in each case a renal panel of different congestive heart failure patients having a GFR of less than 15 mL/min/1.73 m². According to prior art techniques, the calculation of the GFR did not indicate at all the pathophysiologic reasons for such an impaired GFR. Thus, by analyzing blood samples of those patients according to prior art techniques, one would have only obtained the information that they have strongly limited renal function. According to the embodiment, the results of which are displayed in FIGS. 6A to 6C, the information of an impaired GFR is supplemented by detailed information on the pathophysiologic background of the examined patients. Thereby, the renal panel indicates the determined concentrations of nine different substances, namely creatinine, creatine, myo-inositol, glycerol, dimethylamine, dimethyl sulfone, valine, leucine, and isoleucine. The non-filled bars (or histograms) indicated for each substance represent the concentrations of the substances found in patients with normal GFR (eGFR without pathological findings). The filled grey bar ranging from bottom to top of each concentration plot indicates the determined concentration for the respective individual patient.

The patient, the renal panel of which is depicted in FIG. 6A has a myo-inositol concentration of approximately 310 mg/L and a dimethyl sulfone concentration of approximately 25 mg/dL. Myo-inositol is indicative for the presence of uremia. Dimethyl sulfone is indicative for renal oxidative stress. Compared with the myo-inositol and dimethyl sulfone concentrations of the patients the renal panel of whom are represented in FIGS. 6B and 6C, the myo-inositol concentration and the dimethyl sulfone concentration of the renal panel of FIG. 6A are in a medium-range. This is indicated by horizontally arranged double arrows. Thus, the myo-inositol concentration and the dimethyl sulfone concentration of the patient the renal panel of whom is depicted in FIG. 6A are neither particularly high nor particularly low with respect to other patients having a comparably impaired GFR. Thus, neither uremia nor oxidative stress appears to be markedly pronounced in this patient.

Turning now to FIG. 6B, the concentration of myo-inositol is approximately 330 mg/L and the concentration of dimethyl sulfone is approximately 19 mg/dL. Thus, in comparison with the patient the renal panel of whom is depicted in FIG. 6A, the patient, the renal panel of whom is depicted in FIG. 6B, appears to have a more pronounced uremia and even less pronounced oxidative stress. Thus, it is more appropriate to prescribe a therapy for the patient, the renal panel of whom is depicted in FIG. 6B, aiming at relieving uremia than at relieving oxidative stress.

Turning now to FIG. 6C, the concentration of myo-inositol is only approximately 220 mg/L, whereas the concentration of dimethyl sulf one is approximately 39 mg/dL. Thus, the patient, the renal panel of whom is depicted in FIG. 6C, appears to have only a low tendency for uremia, but highly pronounced renal oxidative stress. Thus, the therapy of this patient needs to be directed to lowering the oxidative stress, but not necessarily needs to address alleviations of uremia.

Thus, the patients, the renal panels of whom are depicted in FIGS. 6A to 6C, have similar low GFRs and thus a similar low kidney function. However, the pathophysiologic background of the impaired kidney function is completely different. While the patient, the renal panel of whom is depicted in FIG. 6A neither has a very pronounced uremia nor very pronounced renal oxidative stress, the patient, the renal panel of whom is depicted in FIG. 6B has a pronounced uremia but only low renal oxidative stress. In contrast, the patient, the renal panel of whom is depicted in FIG. 6C has only very low pronounced uremia, but strongly pronounced oxidative stress. Thus, the three patients can be grouped into different groups or classes. Furthermore, their impaired kidney function should be treated by different therapies. This embodiment reveals that the novel biomarkers establish a particularly simple possibility to not only precisely calculate the GFR but to obtain at the same time insight into the pathophysiologic background of impaired GFR and to provide original data on the basis of which physicians can subsequently make decisions on further therapeutic treatments.

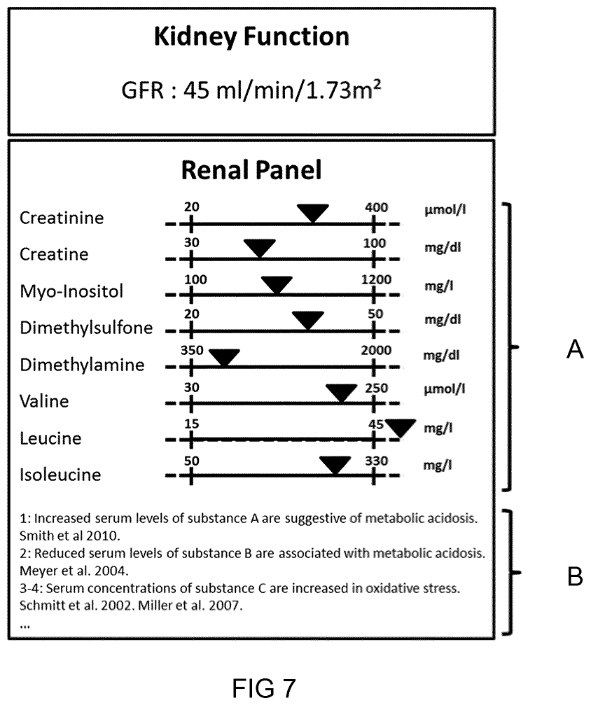

FIG. 7 is an exemplary embodiment of a kidney function report. This report is output to a patient and/or to medical staff in an embodiment of the described methods. In its upper portion, this kidney function report prominently indicates the calculated GFR. In its lower portion, it graphically represents a renal panel. Thereby, it indicates the determined concentrations of different substances, wherein creatinine, creatine, myo-inositol, dimethyl sulf one, dimethylamine, valine, leucine, and isoleucine are chosen in this exemplary embodiment as substances. The concentration of the substances is presented on a scale ranging from lower to upper thresholds indicating expected ranges (or reference ranges) for the examined patients. In some instances, the concentration of one or more substances can also lie outside these ranges. For example, leucine has a concentration of more than 45 mg/L and is thus strongly over-concentrated in the blood of the considered patient.

This output of serum substance concentrations in the form of quantified values in conventional units is presented in section A of the lower portion of the kidney function report. In section B of the lower portion of the kidney function report, the pathophysiological background is explained more detail. In doing so, only pathophysiologic context from literature relating to those substances the concentration of which is significantly increased or decreased in the examined patient is listed. In an embodiment, also the pathophysiologic context from literature relating to substances the concentration of which is not significantly increased or decreased can be listed.

### Exemplary Embodiment: Evaluation of Different Marker Subsets

Different marker subsets comprising at least substances from the list of possible substances have been built. Afterwards, the performance of the subsets has been tested in detail. The performance was compared with the performance of eGFR calculation methods known from prior art, namely eGFR calculations based on creatinine (eGFR_{creat}) and based on cystatin C (eGFR_{cysC}). In the following Table 1, the so-called P30 value is represented. It indicates the 30% confidence interval. Thus, a P30 value of 71% indicates that the eGFR differs in 71% of the examined samples (patients) at a maximum of 30% from the real (measured) GFR.

TABLE 1
P30 values of different models for calculating the eGFR.

| | P₃₀ value |
|---|---|
| | with respect |
| Model for calculating the eGFR | to mGFR |
| eGFR_{creat} (Comparative example) | 71% |
| eGFR_{cysC} (Comparative example) | 60% |
| eGFR (creatinine, dimethyl sulfone, valine) | 72% |
| eGFR (creatinine, myo-inositol, valine) | 78% |
| eGFR (creatinine, myo-inositol, valine, | 81% |
| dimethyl sulfone) | |
| eGFR (creatinine, creatine, dimethyl | 84% |
| sulfone, glycerol, valine) | |

The P30 values of all four subsets of the novel biomarkers listed in Table 1 are higher than the P30 values of the eGFRs calculated according to established methods based on the serum creatinine level or based on the serum cystatin C level.

When estimating the GFR based on plasma clearance by tracer injection and taking a blood sample, a P30 value of 86% is achieved. Thus, embodiments of the proposed solution show a performance that equals the performance of plasma clearance but do not require the injection of any tracer substances. Thus, the presently described method significantly facilitates the precise analysis of renal function and allows reliable results giving optionally additional insight into the pathophysiologic background of the detected impaired kidney function.

1. An in vitro method for calculating a glomerular filtration rate of an individual or an indicator of a pathophysiologic factor of an. impaired glomerular filtration rate, the method comprising the following steps:
a) providing a biocd sample from an individual:
b) determining the concentration of at least three substances chosen from the group consisting of alaine, choline, creatine, creatinie, dimethyl, sulfone, dimethylamine, glucose, glycerol, isoleucine, leucine, myoinositol, N,N-dimethylglycine, and valine in the blood sample by analyzing the blood sample with a suited measuring technique; and
c) calculating a predicted giomeroiar filtration rate or an indicator of a pathophysiologic factor of an impaired glomerular filtration rate from the determined concentrations of the substances,
wherein a murker set comprises the at least three substances.

2. The method according to claim 1, wherein the group consists of creatine, creatinine, dimethyl sulfone, glycerol, myo-inositol, and valine.

3. The method according to claim 1, wherein the marker set comprises creatinine, dimethyl sulfone, and valine.

4. The method according to claim 1, wherein the marker set comprises creatinine, myo-inositol, and valine.

5. The method according to claim 1, wherein the marker set comprises creatinine, dimethyl sulfone, myo-inositol, and valine.

6. The method according to claim 1, wherein the marker set comprises creatine, creatinine, dimethyl sulfone, glycerol, and valine.

7. The method according to claim 1, wherein the pathophysiologic factor is at least one of renal failure, renal co-morbidity, and extra-renal co-morbidity.

8. The method according to claim 1, wherein the pathophysiologic factor is at least one of the group consisting of altered renal filtration, reabsorption and/or secretion capability, uremia, renal metabolic acidosis, renal oxidative stress, altered renal gluconeogenesis, amyotrophia, creatinine to muscle mass ratio, changes of gut microbiome and/or tubular hyperosmolality.

9-10. (canceled)

11. The method according to claim 1, wherein the predicted glomerular filtration rate is calculated by applying at least one of the following equations:
a) GFR=a−b log(substance 1)−c log(substance 2)*d log(substance 3) Equation 3
wherein
a is a number in the range of 100 to 260;
b is a number in the range of 10 to 100;
c is a number in the range of 1 to 10;
d is a number in the range of 10 to 100; and
(substance n) denotes the concentration of the respective substance n;
b) GFR=exp(f)*(substance 4)⁻g* (substance 5)^{-h}*(substance 6)ⁱ Equation 4
wherein
f is a number in the range of 1 to 20;
g is a number in the range of 0.1 to 2.0;
h is a number in the range of 0.1 to 2.0;
i is a number in the range of 0.1 to 2.0; and
(substance n) denotes the concentration of the respective substance n;
c) GFR_{A}=exp(−j)*(substance 7)^{-k}*(substance 8)^{l}*(substance 9)^{m}* (substance 7)^{o log(substance 8),} Equation 5
GFR_{B}=p−qlog(substance 7)−r log(substance 10) Equation 6
wherein
j is a number in the range of 0.1 to 10;
k is a number in the range of 0.1 to 2.0;
l is a number in the range of 0.1 to 10;
m is a number in the range of 0.1 to 2.0;
o is a number in the range of 0.1 to 2.0;
p is a number in the range of 100 to 360;
q is a number in the range of 10 to 100;
r is a number in the range of 1 to 40;
(substance n) denotes the concentration of the respective substance n;
GFR_{A} denotes the predicted GFR for a first GFR range and
GFR_{B} denotes the predicted GFR for a second GFR range;
GFR_{A}=exp(s)*(substance 11)^{t}* (substance 12)^{u}* (substance 11)^{-v log(substance 13)}, Equation 7
d) GFR_{B}=w−xlog(substance 14)−y log(substance 15)−z log(substance 14)* log(substance 13) Equation 8
wherein
s is a number in the range of 0.1 to 10;
t is a number in the range of 0.1 to 10;
u is a number in the range of 0.1 to 2.0;
v is a number in the range of 0.1 to 2.0;
w is a number in the range of 50 to 220;
x is a number in the range of 10 to 100;
y is a number in the range of 1 to 40;
z is a number in the range of 1 to 40;
(substance n) denotes the concentration of the respective substance n;
GFR_{A} denotes the predicted GFR for a first GFR range and
GFR_{B} denotes the predicted GFR for a second GFR range.

12. The method according to claim 1, wherein at least a first equation and a second equation are combined for calculating the predicted glomerular filtration rate, wherein the first equation is optimized for a first glomerular filtration rate range, and the second equation is optimized for a second glomerular filtration rate range, and wherein the first glomerular filtration rate range and the second glomerular filtration rate range are spaced apart from each other, adjoin or partially overlap.

13. The method according to claim 12, wherein the first glomerular filtration rate range comprises glomerular filtration rates indicative for hypofiltration, and wherein the second glomerular filtration rate range comprises glomerular filtration rates indicative for hyperfiltration or normal glomerular filtration.

14. The method according to claim 12, wherein the first equation is weighted by a first weighing factor and the second equation is weighted by a second weighing factor, wherein a) the first weighing factor is bigger than the second weighing factor if the predicted glomerular filtration rate, when calculated using the non-weighted first equation and/or the non-weighted second equation, falls within the first glomerular filtration rate range but not within the second glomerular filtration rate range, or b) the first weighing factor is smaller than the second weighing factor if the predicted glomerular filtration rate, when calculated using the non-weighted first equation and/or the non-weighted second equation, falls within the second glomerular filtration rate range but not within the first glomerular filtration rate range.

15. The method according to claim 12, wherein a third equation is combined with the first equation and the second equation, wherein the third equation is optimized for a third glomerular filtration rate range, wherein the first glomerular filtration rate range, the second glomerular filtration rate range, and the third glomerular filtration rate range are spaced apart from each other, adjoin or partially overlap with at least one of the respective other glomerular filtration rate ranges.

16. The method according to claim 1, wherein a classification of the individual, the blood sample of whom is analyzed, into a predefined class is performed based on a pattern of the determined concentrations of the substances.

17. The method according to claim 16, wherein the classification is supplemented by an indication of a likelihood that the individual is in transition to another predefined class.

18. The method according to claim 1, wherien in case that the calculated glomerular filtration rate is indicative of hypofiltration or hyperfiltration, the determined concentrations of the substances are additionally used to provide an indicator of a pathophysiologic factor of an impaired glomerular filtration rate, wherein the determined concentration of at least one substance is compared with the concentration of the same substance in the blood of another individual having a similar glomerular filtration rate.

19. The method according to claim 18, wherein the pathophysiologic factor is at least one of renal failure, renal co-morbidity, and extra-renal co-morbidity.

20. The method according to claim 18, wherein the pathophysiologic factor is at least one of the group consisting of altered renal filtration, reabsorption and/or -secretion capability, uremia, renal metabolic acidosis, renal oxidative stress, altered renal gluconeogenesis, amyotrophia, creatinine to muscle mass ratio, changes of gut microbiome and/or tubular hyperosmolality.

21. The method according to claim 18, wherein the indicator of a pathophysiologic factor of an impaired glomerular filtration rate and serves as basis for at least one of assisting in assessing the eligibility of diabetic patients for metformin therapy;
assisting in evaluating a contribution of kidney dysfunction to edema formation for congestive heart failure patients; supporting a diagnosis of kidney disease in children; assisting in identifying suitable candidates for live kidney donation; assisting in evaluating kidney function after renal allograft transplantation; supporting an evaluation of the eligibility of chronic kidney disease stage IV patients to enter kidney transplant waiting list; assisting in evaluating the risk for acute kidney injury peri liver transplantation and/or post liver transplantation; assisting GFR estimation in patients having a body constitution significantly differing from the body constitution of a healthy standard population; assisting in evaluating the necessity of liver and kidney co-transplantation;
and assisting in adapting a drug dose administered to a patient.

22. A marker set comprising at least three substances chosen from the group consisting of alanine, choline, creatine, creatinine, dimethyl sulfone, dimethylamine, glucose, glycerol, isoleucine, leucine, myo-inositol, N,N-dimethylglycine, and valine for use in diagnostics of renal function.
